(19) United States
(12) Patent Application Publication
(10) Pub. No.: US 2025/0127397 A1
(43) Pub. Date: Apr. 24, 2025

(54) Intraocular Pressure Sensor

(71) Applicant: Board of Trustees of Michigan State University, East Lansing, MI (US)

(72) Inventors: Li WEN, East Lansing, MI (US); Arthur J. WEBER, East Lansing, MI (US)

(73) Assignee: Board of Trustees of Michigan State University, East Lansing, MI (US)

(21) Appl. No.: 18/935,762

(22) Filed: Nov. 4, 2024

Related U.S. Application Data
(63) Continuation of application No. PCT/US2023/021660, filed on May 10, 2023.
(60) Provisional application No. 63/340,619, filed on May 11, 2022.

(51) Int. Cl. A61B 3/16; H01Q 1/22; H01Q 7/00

(52) U.S. Cl. CPC A61B 3/16; H01Q 1/2225; H01Q 7/00; A61B 2562/0247; A61B 2562/0271; A61B 2562/12

(57) ABSTRACT

A pressure sensor apparatus is provided. In another aspect, a wireless intraocular pressure sensor includes a deformable or stretchable inductor having a three-dimensionally serpentine or wavy shape. A further aspect of an intraocular pressure sensing system includes a closed loop, deformable and variable inductor of an undulating shape in lateral and depth directions, within a ring-shaped and polymeric carrier layer, sized to contact an eye. A method of making a wireless intraocular pressure sensor, including a three-dimensionally deformable metal layer, is also provided.

### CROSS-REFERENCE TO RELATED APPLICATIONS

The present application is a continuation of PCT Patent Application Serial No. PCT/US2023/021660, filed on May 10, 2023, which claims priority to U.S. Provisional Patent Application Ser. No. 63/340,519, filed on May 11, 2022, both of which are incorporated by reference herein.

### BACKGROUND AND SUMMARY

The present disclosure relates generally to pressure sensors and more particularly to a wireless intraocular pressure sensor system and method for making same.

Glaucoma is the second leading cause of blindness which is an asymptomatic, progressive and irreversible disease that is usually associated with elevated intraocular pressure. Most conventional constructions of intraocular pressure sensors can be categorized into three general groups in terms of their energy transferring mechanisms: active, passive, and radio-frequency-powered devices.

Active devices traditionally employ application specific integrated circuits that can store, process and transmit data. However, they make the overall device large, heavy and inflexible. Furthermore, such active systems typically require an integrated battery or power-receiving coil, which both add to the weight and size of the device.

Traditional passive sensors require high precision surgery to anchor the device to the iris. The fabrication process for such passive devices is typically complex and expensive. Furthermore, such traditional passive sensors require an external reading mechanism of an undesirably large size that interferes with the vision of the patient thereby making it unsuitable for wearable long term measurements.

Conventional radio frequency or electromagnetic coupled sensors have employed variable capacitors to sense pressure. These variable capacitors, however, require a pressurized reference chamber that has great difficulty in sustaining its baseline pressure over time due to packaging imperfections. This causes significant undesirable signal drift due to the leakage.

Exemplary intraocular pressure sensors are disclosed in: U.S. Patent Publication No. 2017/0280997 entitled “Non-Invasive Intraocular Pressure Monitor” which published to Lai et al. on Oct. 5, 2017; U.S. Patent Publication No. 2016/0051144 entitled “Systems and Methods for Monitoring Eye Health” which published to Rickard et al. on Feb. 25, 2016; U.S. Patent Publication No. 2012/0277568 entitled “Wireless Intraocular Pressure Monitoring Device, and Sensor Unit and Reader Unit Thereof” which published to Chiou et al. on Nov. 1, 2012; U.S. Pat. No. 9,289,123 entitled “Contact Lens for Measuring Intraocular Pressure” which issued to Weibel et al. on Mar. 22, 2016; and WO 2016/071253 entitled “Physiological Parameter Monitoring Device” which published to Moreau et al. on May 12, 2016. All of these are incorporated by reference herein. It is noteworthy that Weibel includes a considerable quantity of electronic components. Moreover, Rickard uses only capacitive sensing and requires surgical implantation into the eye. All of these traditional constructions, however, do not allow for sufficient sensor expansion to provide sensitive enough, intraocular pressure sensing and/or are overly complex to manufacture.

Furthermore, commonly owned U.S. Patent Publication No. 2020/0015678 entitled “Intraocular Pressure Sensor,” which published to common co-inventors Li and Weber on Jan. 16, 2020, provides significantly improved sensor configurations. This patent publication is incorporated by reference herein. However, further refinements in shape and manufacturing techniques are desired to enhance flexibility for manufacturing.

In accordance with the present invention, a pressure sensor apparatus is provided. In another aspect, a wireless intraocular pressure sensor includes a deformable or stretchable inductor having a three-dimensionally serpentine or wavy shape. A further aspect of an intraocular pressure sensing system includes a closed loop, deformable and variable inductor of an undulating shape in lateral and depth directions, within a ring-shaped and polymeric carrier layer, sized to contact an eye. Another aspect provides an organ pressure sensing system including a passive inductor with a three-dimensional wavy, undulating or serpentine shape that allows for lateral and depth flexibility and stretching during manufacturing, such as while being encapsulated. A method of making a wireless intraocular pressure sensor, including a three-dimensionally deformable metal layer, is also provided.

The present pressure sensor is advantageous over conventional devices. For example, the present three-dimensional serpentine shape of the antenna and inductor provides superior flexibility and stretchability during encapsulating molding within a polymeric carrier, thereby deterring undesired fracturing and damage during high volume manufacturing. The present pressure sensor functions as a passive strain gauge that synergistically serves as both a pressure sensitive element and a wireless communications interface. It advantageously has a central aperture during extended use such that it does not obstruct the vision of the patient, as compared to conventional cornea-mounted devices. Furthermore, in one exemplary ophthalmological construction, the present system is intended to be a temporarily worn device that is easily removable after a predetermined period of time, and does not require surgical in vivo implantation or removal. Moreover, the present system advantageously includes only minimal electronic components, such as a single capacitor, located in the sensor device inserted onto the eye; this provides a much lighter weight and lower cost device which does not obstruct the patient's vision, while providing a single closed loop, multifunctional antenna and inductor, thereby decreasing lateral size. Additional advantages and features of the present system and method will become apparent from the following description and appended claims, taken in conjunction with the associated drawings.

### BRIEF DESCRIPTION OF THE DRAWINGS

FIG. 1 is a front elevational view showing a preferred embodiment of an inductor antenna and capacitor employed in the present intraocular pressure sensor apparatus;

FIG. 2 is a cross-sectional view, taken along line 2-2 of FIG. 6, showing the present intraocular pressure sensor apparatus;

FIG. 3 is a cross-sectional view, taken along line 3-3 of FIG. 6, showing the present intraocular pressure sensor apparatus;

FIG. 4 is a fragmentary side elevational view, taken along arrow 4 of FIG. 1, showing the present intraocular pressure sensor apparatus;

FIG. 5 is a cross-sectional view, like that of FIG. 2, showing an intermediate manufacturing configuration of the present intraocular pressure sensor apparatus;

FIG. 6 is a front elevational view showing the final manufactured, present intraocular pressure sensor apparatus with an encapsulating carrier;

FIG. 7 is a perspective view showing the final manufactured, present intraocular pressure sensor apparatus;

FIG. 8 is a fragmentary perspective view showing a second configuration of the present intraocular pressure sensor apparatus;

FIG. 9 is a diagrammatic side view showing the present intraocular pressure sensor apparatus mounted to a patient's eye;

FIG. 10 is an electrical circuit diagram showing the present intraocular pressure sensor apparatus;

FIGS. 11-13 are perspective views showing the first manufacturing tools and process for making the present intraocular pressure sensor apparatus;

FIGS. 14A-J are a series of diagrammatic side views showing a second manufacturing process for making the present intraocular pressure sensor apparatus;

FIG. 15 is a front elevational view showing a third configuration of the final manufactured, present intraocular pressure sensor apparatus with an encapsulating carrier;

FIG. 16 is a perspective view showing the third configuration of the final manufactured, present intraocular pressure sensor apparatus; and

FIG. 17 is a diagrammatic diagram showing a fourth configuration of the present intraocular pressure sensor apparatus.

### DETAILED DESCRIPTION

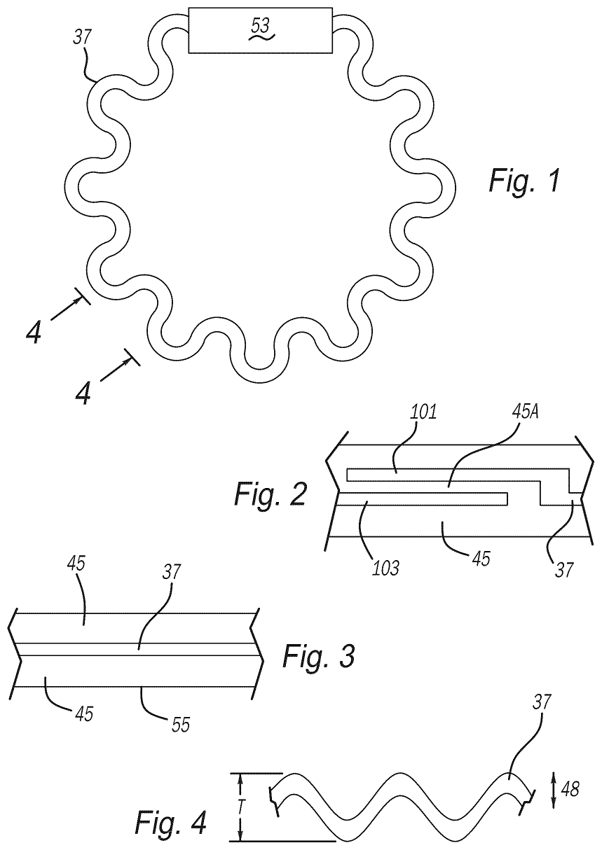

A first embodiment of an intraocular pressure sensing system 31 is shown in FIGS. 1-9, and includes an intraocular pressure sensor 33 and a reader 35. Sensor 33 includes a sensing and transmitting coil assembly 37, and an optional polymeric carrier 39. Coil assembly 37 has a looped metallic inductor 43 at least partially, and preferably, fully encapsulated within a protective polymeric layer or casing 45.

Inductor 43 has a three-dimensional undulating, serpentine and wave-like shape along a curved longitudinal length direction 46 thereof (see FIG. 6) and along a perpendicular thickness direction 48 thereof (see FIG. 4), defined by alternating peaks and valleys with curved diagonal walls connecting therebetween in both perpendicular longitudinal and thickness planes. The undulating shape allows inductor 43 to be deformed and stretchably expanded at any point therealong both during manufacturing and also during patient use.

Inductor 43 is preferably made from layers of titanium and copper, titanium and gold, or an alloy thereof. Titanium acts as an adhesion promoter. Alternately, the metallic material may be a single layer of gold or copper. Alternately, other metallic or conductive polymeric materials may be employed although they may not be as advantageous; for example, Ti and Chromium may be used. The polymeric material for protective layer 45 of sensor 33 is preferably Parylene-C (obtained from Parylene Coater-Specialty Coating System as PDS 2010), Sylgard® 184 Silicone Elastomer (obtained from Dow Corning), Polymethyl Methacrylate (obtained from MicroChem Corporation), polyimide, or the like. Parylene-C is a poly-para-xylylene polymer modified by the substitution of a chlorine atom for one of the aromatic hydrogens; it is a linear and highly crystalline material this has dielectric properties with a very low moisture permeability.

A single electrical component, preferably a capacitor 53, is electrically connected to ends of inductor 43 and secured to sensing coil assembly 37. This is preferably the only electronic component directly attached to sensor 33, such that a battery, microprocessor and other solid state electrical components are not required, thereby saving weight and reducing cost.

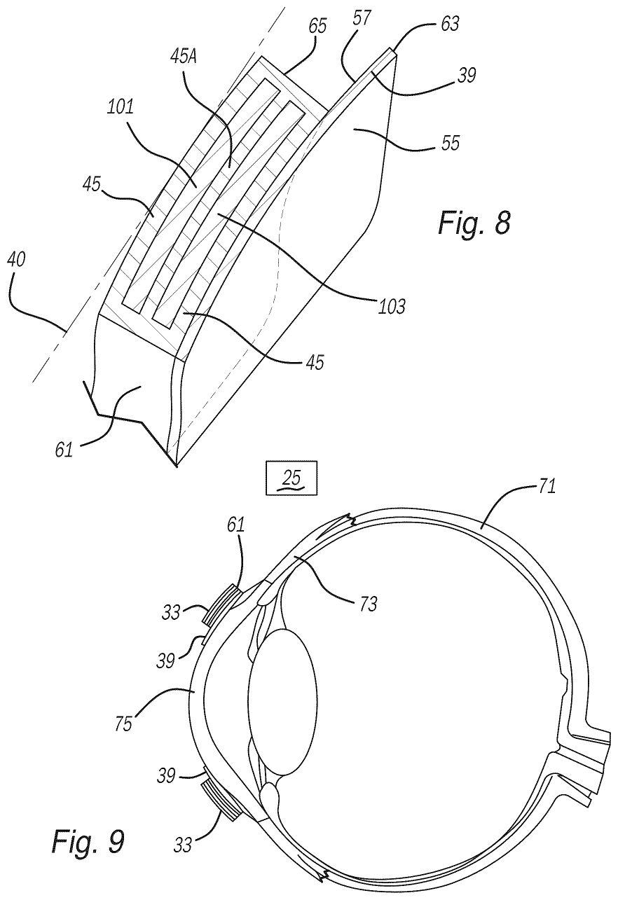

Carrier 39 is three-dimensionally curved relative to a lateral plane 40, with a donut-shaped and annular true view shape, having an eye-contacting internal surface 55 and an opposite external surface 57, upon which is coupled sensor 33. Carrier 39 is a flexible polymeric material such as a hydrophilic thermoplastic parylene. Sensor 33 is preferably entirely encased and encapsulated within carrier 39. Protective polymeric layer 45 of sensor 33 has a generally common circular inner diameter edge 61 with carrier 39, but an outer diameter edge 63 of the carrier optionally extends radially and laterally past and is larger than an outer diameter edge 65 of the protective layer of the sensor, as can best be observed in FIG. 8. An exterior peak surface to opposite exterior valley surface dimension T is about 100-200 microns, and more preferably 150-200 microns. Alternately, however, sensor 33 may be adhesively bounded upon external surface 57 of carrier.

The placement of sensor 33 relative to an organ, specifically a patient's eye 71, can best be observed in FIGS. 8 and 9. Interior surface 55 of carrier 39 is temporarily mounted to cornea 75 by a dissolvable adhesive such as a hydrogel based adhesive. Carrier 39 and sensor 33 mounted thereon are all internal to a sclera 73 of eye 71. Nevertheless, an optional dissolvable polymeric film, of a generally dome-like shape, spans between inner edge 61 of sensor 33 and/or carrier 39, and initially spans across a center of cornea 75. This optional central film is simply intended to add temporary supporting structure to the thin and deformable sensing coil assembly during user or doctor insertion onto the eye and is intended to dissolve away within a day or two thereafter such that there is an unobstructed central opening over the cornea during normal sensing use. Protective layer 45, carrier 39 and the optional central film are all substantially transparent.

Reference should now be made to FIGS. 1-4 and 6 for another exemplary configuration for capacitor 53. Ends 101 and 103 of inductive coil 37 extend past each other such that they overlap each other in a stacked but spaced apart manner. An intermediate portion 45A of encapsulating layer 45 serves as a predominantly non-conductive insulator between the overlapped ends of inductor 37. Thus, overlapping inductive ends 101 and 103 function as a capacitor integrated as a synergistic and multifunctional construction along with the sensing inductance and signal transmitting antenna-like functions. This integral and overlapping microfabricated version advantageously eliminates the separate capacitor assembly steps and improves device reliability and production yield. Accordingly, a separate capacitor component is not required.

In all of these embodiments, inductive coil 37 with either its overlapping capacitive ends or with the separately mounted capacitor component, defines a single closed loop shape, without a spiral or multiple concentric coils, when seen in a true view like in FIG. 6. It should be appreciated that while it is preferred to use this integrated inductor and capacitor device for a wireless intraocular pressure sensor, it should also be appreciated that this device may alternately be applied to a variety of other types of biomedical sensors that are attached to or implanted in a patient.

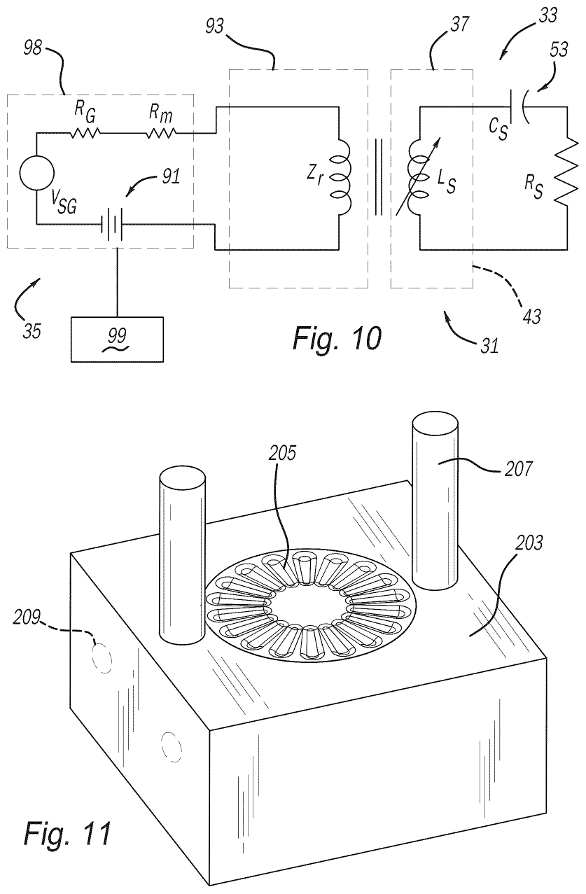

Referring to FIGS. 9 and 10, reader 25 is preferably mounted to eyeglasses which include transparent lenses, a frame and earpieces. Reader may alternately be placed in a shirt pocket or neckless worn by the patient. Reader 35 is remotely located away from but within 25 cm, and more preferably within 5 cm, of sensor 33.

A reading coil or wire is encapsulated within or adhered to an inside surface of eyeglass frame generally surrounding each lens. Ends of the looped reading coil are electrically connected to an electrical circuit including a battery for a wearable/portable reader or other power supply 91 accessed through a wall outlet plug for a stationary reader, a signal generator voltage source V_{SG}, a signal generator internal resistor R_{G}, and a measurement resistor R_{M}. In the circuit diagram, reading coil 93 is shown as a receiver coil impedance inductor Zᵣ. Furthermore, constant capacitor 53 is shown as C_{S}, a parasitic resistor function of the inductor is shown as R_{S}, and variable inductor 37 is illustrated as L_{S}. Alternately, the reader coil may be attached to a fabric sleep mask, removably covering the eyes and with an elastic head band, for continuous IOP monitoring when the patient is sleeping.

Periodic electromagnetic transmissions are sent from the reader coil to the passive sensor coil to activate a resonant frequency of the sensor coil. This resonant frequency is based on the geometries of the inductor and capacitor, and has an exemplary frequency of 100 MHz to 1 GHZ, which can be varied depending on the detectable frequency range of the impedance analyzer. A frequency shift of the phase dip is observed when the inductor is deformed. The phase dip is an indicator of the resonant frequency such that when the sensing inductor is deformed in response to pressure variation, a frequency shift of this phase dip can be detected.

If intraocular pressure outwardly bulges or expands sclera 73 of eye 71 then sensor 33 will move off its nominal position. This movement will subsequently deform, flex or circumferentially expand inductor 43 away from its nominal free position which will accordingly change an inductance value received by reader coil 93 from inductor 43. The sensor serves as a planar and circular LC passive resonator that has a constant capacitor and a stretchable variable inductor. The self-inductance of the inductor, the parasitic capacitance between the segments, and the Q-factor are all changeable by the expansion of the sensor diameter. In addition, the mutual inductance between the sensing coil and reader coil will also change as the sensing coil deforms. This phenomenon is employed to measure the strain and the eye tissues induced by intraocular pressure elevation. Accordingly, the change of self-inductance in parasitic capacitance results in the change of resonance frequency of the LC loop. Therefore, the pressure variance can be read by the impedance analyzer through a frequency drift. The resultant output from an impedance analyzer 98 of reader 35 is transmitted via communications transmitter 99 to a handheld or stationary computer device, through a Bluetooth standard, a Wi-Fi standard, an RFID standard, a ZigBee standard or the like. Hence, the inductor synergistically functions as both an inductive sensor and a communications antenna, but in a very small and light weight package since it preferably only has a single closed loop coil. An SD card may also be connected to the reader electronic circuit for temporary data storage sent from the sensor.

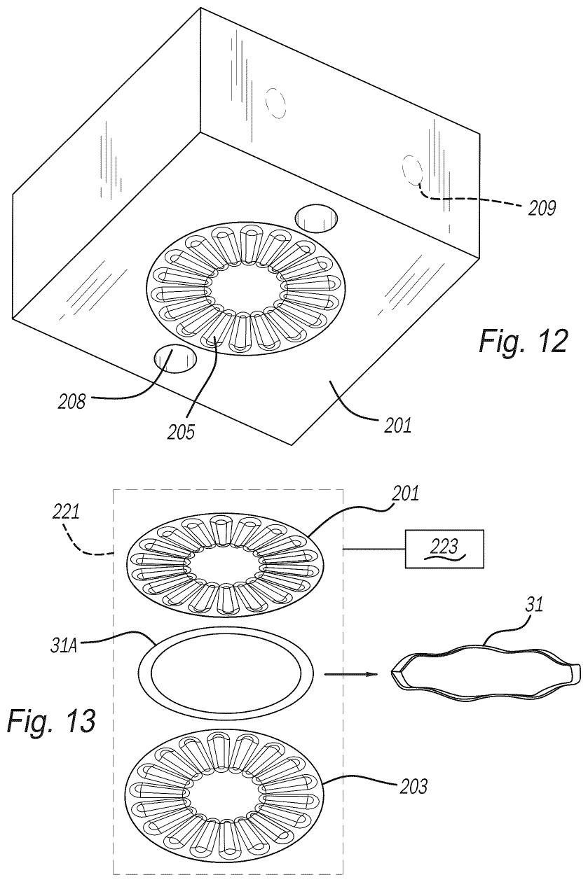

A first exemplary manufacturing or fabrication process for the sensing coil assembly is illustrated in FIGS. 11-13. This version constructs a three dimensionally undulating intraocular pressure sensor with enhanced radial stretchability by using a thermal molding process. First, a flat sensor assembly 31A is microfabricated on a machine platform or substrate surface to have only a two-dimensional undulation in only the longitudinal and radial/lateral directions, while inductive coil 37 is being encased or encapsulated within protective polymeric layer 45 (see FIG. 3). Second, the flat sensor assembly 31A is thereafter formed into a three-dimensionally undulating sensor assembly 31 in a thermal molding process. Then a third step includes adhering together or encapsulating the protective layer and inductor sensor assembly 31 to create the completed IOP sensor apparatus.

More specifically, the two-dimensional inductor and the protective layer, sensor assembly of the preceding first step is created as follows in this embodiment. A polymer is deposited in a chemical vapor deposition process. A glass (or silicon) wafer or substrate is coated with about 1-5 μm of parylene-C polymer, followed by thermal evaporation of a 20 nm layer of titanium and a 200-700 nm layer of gold (or copper). which will become the inductor coil. A photoresist layer or first mask is used to pattern the undulating shape using ultraviolet photolithography and thereafter wet-etching to define the shape of the inductor coil. Thereafter, another 0.5-1.5 μm parylene-C is deposited on the metallic layer, and selectively etched using dry oxygen plasma to create the via through the parylene-C layer with a second photoresist layer mask. Subsequently, a second layer of 20 nm titanium and 700 nm gold (or copper) is thermally deposited and chemically patterned with a third photoresist layer mask to form the top capacitor plate. The capacitor is a double plate capacitor with the first and second metallic layers as the capacitor plates, and 0.5-1.5 μm parylene-C as the dielectric layer. After that, another approximately 1-5 μm polymeric top coating is applied to fully encapsulate the inductor layers. A layer of aluminum (or copper) is deposited on top of the outermost polymeric layer and patterned using a fourth photoresist layer mask to form an aluminum (or copper) mask that protects the sealed metallic inductor core during subsequent plasma dry etching of the polymeric casing to create the trimmed form. Then, the two-dimensional sensing coil assembly 31A is separated from the glass (or silicon) substrate and with the aluminum (or copper) mask removed

The second molding step is discussed in more detail as follows. Upper and lower mold halves 201 and 203, respectively, are machined from aluminum or stainless steel to create matching mirrored image, three-dimensionally undulating cavities 205 therein. Alignment pins 207 and receptacles 208, and optional heating or cooling fluid conduits 209, may also be machined therein or assembled thereto.

Two-dimensionally undulating sensor assembly 31A is placed in a cavity of a mold half, then the mold halves are clamped together to compress the sensor assembly therebetween. The mold halves are located within an oven 221 connected to a vacuum pump 223 which creates a negative pressure vacuum therein of about −22 to −23 inHg. Alternately an inert gas such as nitrogen or helium can be inserted into the oven instead of using a vacuum. The objective is to deter oxidation during thermoforming in order to prevent brittleness of the inductive coil when being shaped.

The mold halves are mounted within a hydraulically driven press such that one mold half is movably clamped against the other during the molding, and then retracted to allow access to the cavities. The mold halves are heated to about 160-180° C. and then compress the sensor assembly for about 1-5 hours, and more preferably 3-5 hours. This temperature range and time do not melt the parylene-C polymeric layer since the temperate falls between its glass transition temperature (approximately 80-90° C.) and its melting point (approximately 290-300° C.). In one example, the compression force exerted on the sensor assembly merely relies on the weight of the upper mold half without more, when the molds are vertically stacked on each other; this allows for a gradual deformation of the inductive coil metal. Nevertheless, alternate combinations of compression forces, temperatures and time may be employed depending on the materials and sizes of the sensor parts. Optionally, a slightly enlarged recess may be provided in one or both of the mold halves to receive the capacitor therein such that it is not compressed during mold closure. It is also envisioned that an alignment structure, such as a small pin or upstanding wall, can be provided in the cavities to ensure accurate placement of the sensor assembly in the mold. This process beneficially molds a three-dimensionally undulating, wavy and serpentine shape to sensor assembly 31 which allows for the inductive coil to stretch in all three X, Y and Z directions, without fracture, when subsequently incorporated into carrier.

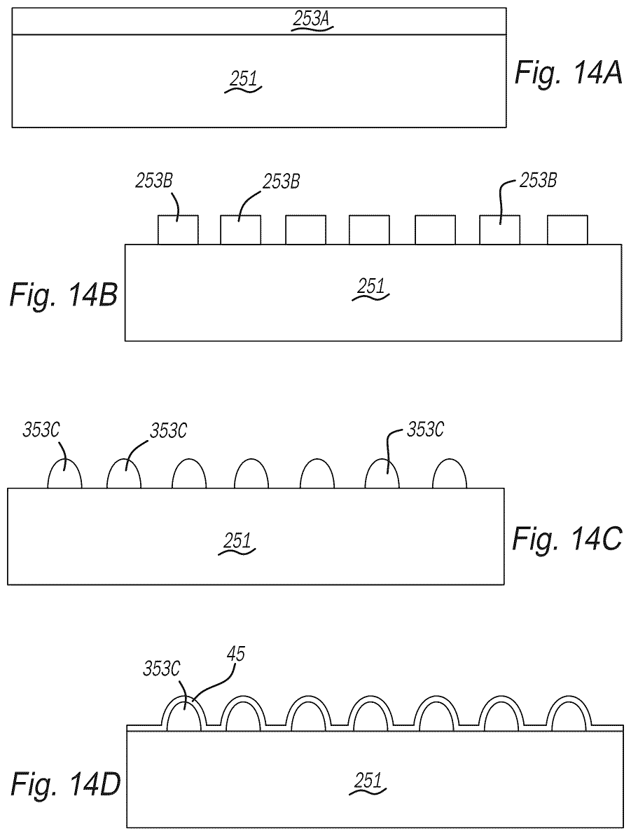

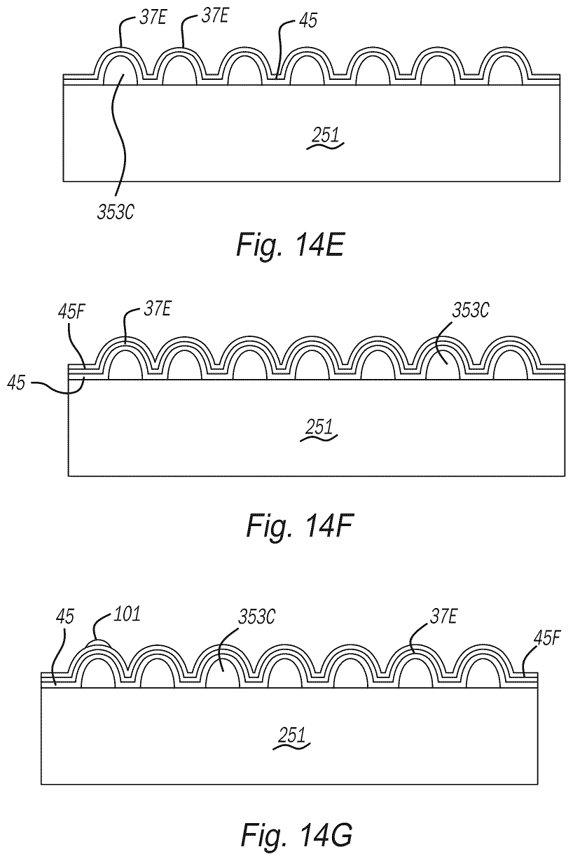

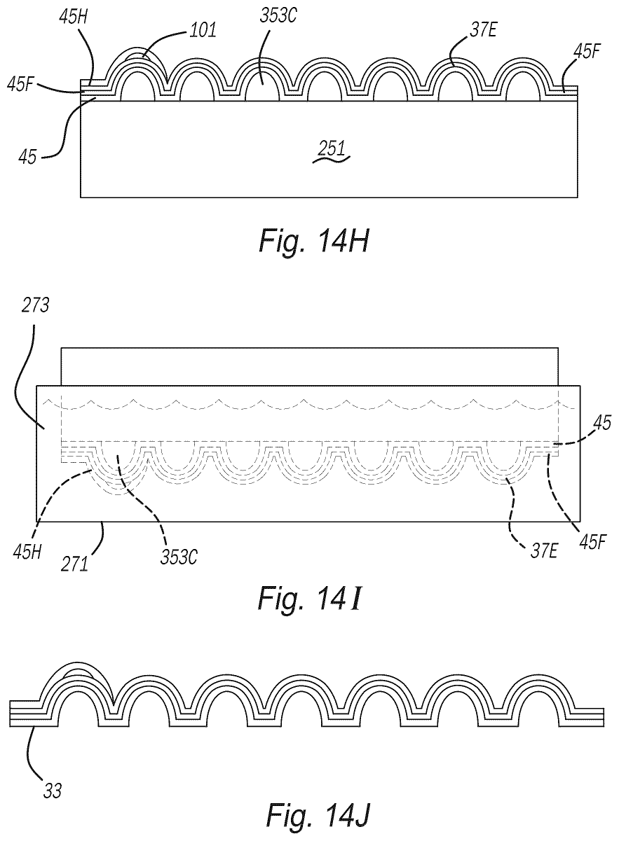

Another method for creating an inductive IOP sensor assembly, having a three-dimensionally undulating, wavy and serpentine shape, will now be discussed with reference to FIGS. 14A-14J. In this method, the wavy IOP sensor can be directly microfabricated on a three-dimensionally undulating substrate 251. The 3D substrate is preferably a silicone material which can be made by three-dimensional printing, photoresist thermal reflowing, metal machining, or the like.

In particular, a photoresist material 253A, such as AZ® 4620 material which can be obtained from AZ Electronic Materials, Inc., is spun onto substrate 251, as is illustrated in FIG. 14A. Centrifuge spinning or casting of the liquid photoresist material can be employed for the spinning. A thickness of photoresist material in this initial step is approximately 5-50 microns.

This is followed by ultraviolet lithography using a mask to pattern the photoresist material 253B into spaced apart, generally rectangular and laterally radiating projections, per FIG. 14B. FIG. 14C shows that during a subsequent thermal reflow processing step, the photoresist structures are heated on a hot plate or in an oven to about 175 to 300° C.; this is above the glass transition temperature of the photoresist and heated for a period of about 1-12 hours to allow the reflow of the soft photoresist under surface tension. This serves to curve the squared off corners to create arcuately curved (in cross-section) photoresist supporting structures 353C. Thus, photoresist supporting structures 353C act as a manufacturing tool, with alternating peaks and valleys, upon which to create the three-dimensionally undulating sensor.

Next, the IOP sensor is constructed using the following sequential steps. A parylene-C layer 45 of about 1-5 μm is deposited on wafer substrate 251 at room temperature, using a chemical vapor deposition system. This parylene evaporate deposition or application step can be observed in FIG. 14D. An exemplary CVD machine for this step is model PDS 2010 Labcoter® 2, from Specialty Coating Systems, Inc.

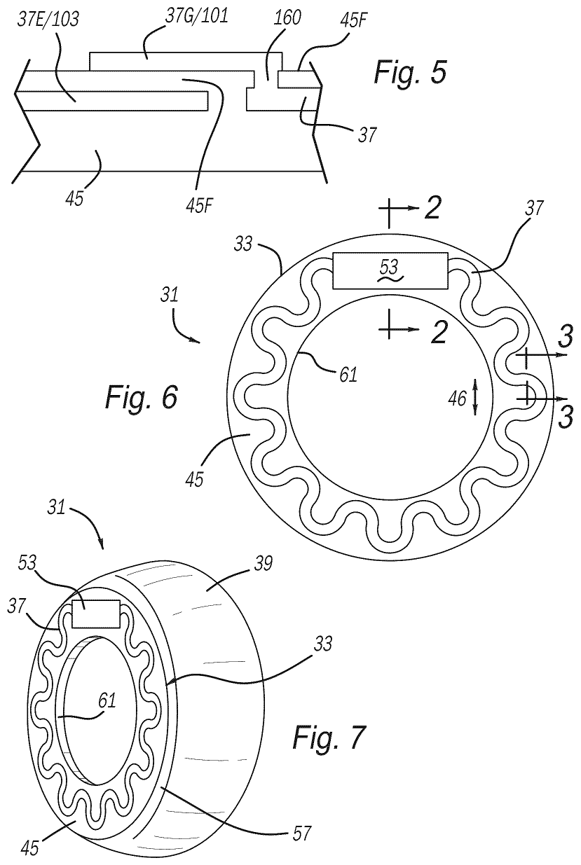

FIG. 14E shows a metallic inductor layer 37E including about 20 nm thick titanium and about 100-700 nm thick gold. This inductor layer 37E is thermally evaporated and deposited on the underlying parylene layer 45 using chemical etching with a photolithographically patterned photoresist mask to form the serpentine metal wire and the lower end section plate 103 (see FIG. 5) of the integrated capacitor. An exemplary machine for such is Auto 306 Thermal Evaporator, from Edward, Inc. Now turning to FIG. 14F, another outermost parylene-C layer 45F, of about 1-5 μm thick, and more preferably 1 μm thick, is applied on top of inductor layer 37E.

Thereafter, FIG. 14G illustrates a layer 101 of about 20 nm thickness of titanium and about 100-700 nm thickness of gold is thermally evaporated and patterned to form the top integrated capacitor section plate 101, and the generally perpendicularly extending neck 160 connecting to nominal inductor layer 37E. The cross-section of FIG. 5 corresponds to this intermediate processing step of FIG. 14G. It is notable that metallic layer 37E is continuous from one edge to the other, serving as a bottom layer of the inductor wire trace, while metallic layer 101 is in a discontinuous manner, such as on only a single peak, acting as the top plate of the double layer capacitor.

FIG. 14H shows a third parylene-C layer 45H, of about 1-5 μm thick, deposited over metallic layer 101. Then, the whole parylene film (including the three overlying layers) is patterned using oxygen plasma dry etching with a copper mask. The copper mask is made by thermally evaporating copper (about 200 nm thick) on the wafer substrate and wet etching to remove unwanted copper with a lithographically patterned photoresist mask.

Referring to FIG. 14I, the layered assembly is dipped into a tank 271 or sprayed with an acetone or developer liquid 273 to remove and dissolve photoresist material 353C. Finally, with regard to FIG. 14J, the freestanding IOP sensor assembly 33 is etched to final trimmed inner and outer peripheral edges using oxygen plasma dry etching with a photoresist mask. The oxygen plasma etching is performed in a reactive ion etcher, such as an RIE-1701 machine from Nordson March, Inc., with a radio frequency power of about 200 W and a processing pressure of about 150 mTorr.

As the third major process, after the sensor device is released from the substrate, a cast molding method is used to embed the sensor into doughnut-shaped soft carrier or lens 39. Two glass molds with concave and convex profiles that match the corneal curvature are used. Initially, sensor 33 is placed on the dome-shaped convex cap and the polymer (for example, HEMA or PDMS) is added to the bottom concave mold. The top convex mold is aligned and pressed into the concave mold, and the thin gap is a cavity between the molds which defines the desired thickness of the combined carrier and sensor apparatus 31. The carrier is subsequently polymerized using either an ultraviolet light process (for HEMA) or thermal process (for PDMS). Following polymerization, the combined carrier and sensor apparatus is release from the concave mold, placed on the convex mold, and the central region of the lens is optionally removed using a tissue punch. Carrier 39 can be manufactured in accordance with U.S. Patent Publication Nos.: 2002/0153623 entitled “Lens Manufacturing Process” which published to Gobron, et al., on Oct. 24, 2002; and 2021/0162692 entitled “Direct Compression Molded Ophthalmic Devices” which published to Rao, et al., on Jun. 3, 2021; both of which are incorporated by reference herein. The doughnut-shaped sensor apparatus 31 then is ready for calibration testing and sterilization.

The sealed sensor attachment to the carrier can include at least one of the following methods: (a) Attaching the sealed sensor to a male mold and thereafter inserting them via immersion into a female mold already containing a carrier polymeric material, in a liquid or semi-liquid state, whereafter the carrier polymeric material is polymerized. (b) Attaching the sealed sensor to a male mold post-coating with a carrier polymeric material, then adding a second liquid top carrier polymeric coating over the entire assembly, and subsequently polymerizing the top polymeric coating. (c) Embedding the sealed sensor into a flat already cured polymer, machining the curved shape of the carrier such as by a lathe, inserting the assembly into a carrier mold, applying additional polymer coatings, finishing the shape with further machining. (d) Inserting the sensor assembly into an injection mold, adding the carrier polymeric material therearound, and polymerizing same.

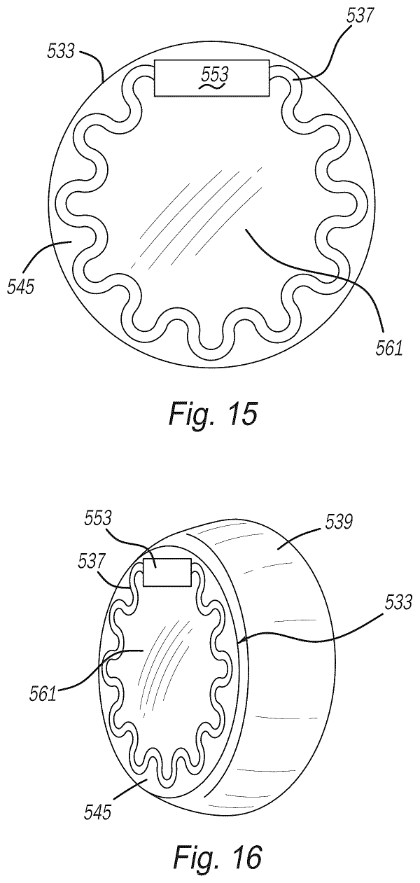

Another embodiment of the present intraocular pressure sensor 533, shown in FIGS. 15 and 16, includes a sensing and transmitting coil assembly 537, and a polymeric carrier 539. Coil assembly 537 has a single looped metallic inductor at least partially, and preferably, fully encapsulated within either a separate protective polymeric layer 545 or integrally within a polymeric contact-lens-like carrier 539. Polymeric layer or carrier includes a transparent and polymeric central portion 561 integrally spanning entirely across the cornea and between the oppositely facing internal edges of coil 537. Therefore, this central portion is preferably not dissolvable and is used instead of the hole defined by inner edge 61 of the FIG. 7 embodiment.

Inductor 543 has a three-dimensional undulating, serpentine and wave-like shape along a curved longitudinal length direction thereof and along a perpendicular thickness direction thereof, defined by alternating peaks and valleys with curved diagonal walls connecting therebetween in both perpendicular longitudinal and thickness planes. A single electronic component, such as a capacitor 553, or overlapping and spaced apart edges of coil 537 (acting in a capacitive manner), are included as part of the sensing and transmitting circuit. This configuration is manufactured in accordance with any of the other embodiments discussed hereinabove.

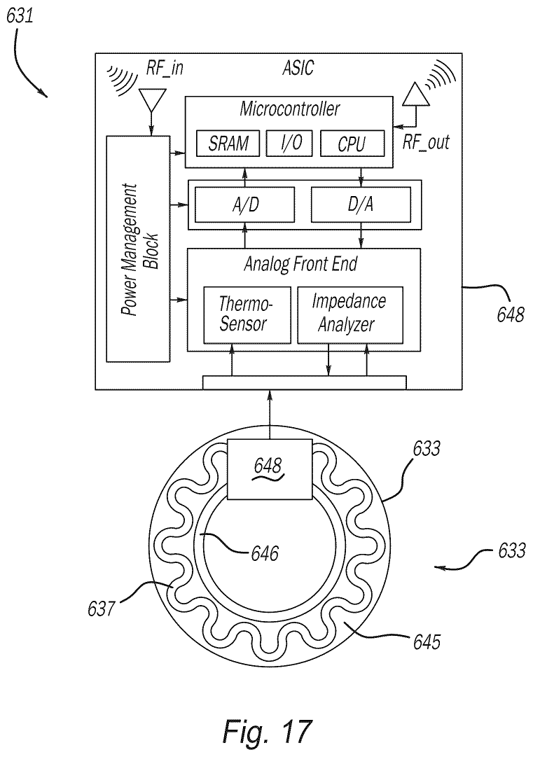

FIG. 17 illustrates yet another embodiment of the present intraocular pressure sensing system 631 including an intraocular pressure sensor 633 and a remote reader. The remote reader may be a handheld electronic module or an eyeglass-mounted module. In this configuration, the three-dimensionally serpentine or wavy inductor is part of an active electrical circuit.

Sensor 633 is preferably of the three-dimensionally undulating shape encapsulated within a polymeric layer 645 and/or carrier as is disclosed hereinabove. The polymeric layer may have either the contiguously central spanning construction or the central hole construction. Intraocular pressure sensor 633 includes an inductive eye pressure sensing coil assembly 637. Additionally, a power sensor and data transmitting antenna 646 is concentrically inside of and medial to coil assembly 637. The three-dimensionally undulating shape of coil assembly 637 will allow for three dimensionally stretching thereof, without fracture, during manufacturing the three-dimensionally curved sensor 633, without requiring the circular and substantially flat shaped power antenna 646 to also have three-dimensional undulations. The benefit of the preferred circular and substantially flat nature of power antenna 646 is that it generates a fixed resonant frequency to sense and transmit both eye temperature and pressure data received from the eye. Optionally, an eye humidity sensing function may be provided in either of the inductor or antenna components.

An ASIC chip 648 is affixed to sensor 633 to process the raw data from power antenna 646 and/or coil assembly 637. In this configuration, coil assembly 637 acts as an active sensor but not a transmitter. This also allows coil assembly 637 to have a greater eye-facing surface area to cover a larger portion of the cornea tissue for greater sensitivity, as compared to the other embodiments set forth herein. The ASIC chip 648 is encased within polymeric layer 645 and includes a microcontroller with SRAM, a CPU and input/output, a thermo-sensor and impedance analyzer, analog-digital converters, a power manager, and a radio frequency receiver and transmitter.

While various embodiments of the present sensor system have been disclosed, it should be appreciated that other variations may be made. For example, alternate electrical circuits and electronic components may be used although some of the present benefits may not be realized. Furthermore, different materials and manufacturing process steps can be used, however, certain of the present benefits may not be achieved. For example, the film coating can alternately be polyimide or PDMS, and Pt for the conducting material, but some of the preferred advantages may not be realized. As another example, the sensing coil assembly and protective layer may be entirely made from biodissolvable material such that the sensor does not need to be manually removed from the eye. The substrate may alternately be glass, metal or ceramic. In another optional configuration, the inductor may have oval or other shapes as long as it forms a generally closed loop in the true view like FIGS. 1, 6, 15 and/or 17 (including the solid state or overlapping ends capacitor to create the closed loop), although some of the desired benefits may not be obtained due to the shape differences. The features of any of the embodiments may be mixed and matched in an interchangeable manner with any of the other embodiments disclosed herein. Various changes and modifications are not to be regarded as a departure from the spirit or the scope of the present invention.

The invention claimed is:

1. An organ pressure sensing system comprising an inductor having a stretchable and three-dimensionally undulating shape which is elongated to create a substantially closed loop, and a polymeric layer surrounding the inductor.

2. The system of claim 1, further comprising a receiver remotely located away from the inductor, the inductor being configured to passively sense internal pressure of an organ to which it is attached, and the receiver being configured to obtain sensed pressure data from the inductor.

3. The system of claim 1, wherein the inductor is a single-loop inwardly spaced from a continuously circular periphery of the polymeric layer, and the inductor is both a pressure sensor and an antenna.

4. The system of claim 1, wherein the polymeric layer is a separate part which is attached to a three-dimensionally curved and polymeric carrier, and the carrier is configured for removeable contact on an eye cornea.

5. The system of claim 1, wherein the polymeric layer is an integral and single piece part with a three-dimensionally curved and polymeric carrier such that the inductor is directly encapsulated within the carrier, and the carrier is configured for removeable contact on an eye cornea.

6. The system of claim 1, wherein there is a central hole in the polymeric layer, surrounded by the inductor.

7. The system of claim 1, wherein the same polymeric layer which encases the inductor, also entirely and contiguously spans across a central area surrounded by the inductor.

8. The system of claim 1, further comprising:
distal ends of the inductor overlapping each other within the polymeric layer and are insulated from each other by an intermediate portion of the polymeric layer being located between the distal ends;
at least one of the distal ends including a step therein to create the overlap; and
all undulating peaks and valleys of the inductor being located along a middle section of the inductor offset from the overlapping distal ends.

9. The system of claim 1, further comprising:
a circular power antenna encapsulated within the polymeric layer, surrounded by the inductor;
a thermo-sensor and an impedance analyzer connected to at least one of the power antenna and the inductor;
a microcontroller including memory and a processor connected to the thermo-sensor and the impedance analyzer; and
a receiver and a transmitter connected to the microcontroller.

10. The system of claim 1, wherein:
a dimension from an exterior peak surface to an opposite exterior valley surface of undulations of the inductor, as measured perpendicular to an organ-contacting surface, is 100-200 microns;
the inductor includes Titanium or an alloy thereof; and
the polymeric layer is dielectric.

11. An organ pressure sensing system comprising:
a single loop inductor having a stretchable and three-dimensionally serpentine shape including multiple peaks and valleys;
a dielectric polymeric casing encapsulating the inductor and having a continuously circular periphery;
the inductor being configured to sense intraocular pressure; and
a dimension from an exterior peak surface to an opposite exterior valley surface of the inductor, as measured in a thickness direction, is 100-200 microns.

12. The system of claim 11, further comprising a receiver remotely located away from the inductor, and the receiver being configured to obtain sensed pressure data from the inductor, and the polymeric casing or a polymeric carrier to which the casing is mounted being removably attached to the organ without in vivo surgery.

13. The system of claim 11, further comprising:
distal ends of the inductor overlapping each other within the polymeric casing and are insulated from each other by an intermediate portion of the polymeric casing being located between the distal ends;
at least one of the distal ends including a step therein to create the overlap; and
all of the undulating peaks and valleys of the inductor being located along a middle section of the inductor offset from the overlapping distal ends.

14. The system of claim 11, further comprising:
a circular power antenna encapsulated within the polymeric casing, surrounded by the inductor;
a thermo-sensor and an impedance analyzer connected to at least one of the power antenna and the inductor;
a microcontroller including memory and a processor connected to the thermo-sensor and the impedance analyzer; and
a receiver and a transmitter connected to the microcontroller.

15. A method of making a pressure sensor, the method comprising:
(a) creating a three-dimensionally undulating shape to an inductor;
(b) encapsulating the inductor within a sensor polymeric material;
(c) placing the sensor in a mold;
(d) adding a carrier polymeric material to the mold to form a three-dimensionally curved carrier configured for removable eye attachment;
(e) stretching undulations of the inductor in three-dimensions during shaping of the carrier without fracturing the inductor;
(f) polymerizing the carrier polymeric material in the mold;
(g) removing the combined carrier and sensor from the mold; and
(h) sterilizing the combined carrier and sensor.

16. The method of claim 15, further comprising making the inductor from a metallic material curved as a single loop and locating the sensor polymeric material so that it has a circular periphery outwardly spaced from the inductor.

17. The method of claim 15, further comprising creating the sensor so it is passive, and the inductor is created to act as both an intraocular pressure sensor and transmitter antenna, and the sensor is configured such that data from the antenna is received by a remotely located reader.

18. The method of claim 15, further comprising encapsulating a circular antenna within the first polymeric material, with the circular antenna being laterally internal to, coaxial with and spaced away from the inductor.

19. The method of claim 15, wherein the inductor is a pressure sensor.

20. The method of claim 15, wherein the inductor is an antenna.

21. The method of claim 15, wherein the creating of the inductor further comprises:
placing a bottom metallic layer in a substantially continuous manner across the inductor;
placing an upper metallic layer in a discontinuous manner on an undulating peak but not an adjacent valley, configured to act as a top plate of the double layer capacitor; and
placing a dielectric polymeric layer between the metallic layers.

22. The method of claim 15, wherein the creating of the inductor comprises depositing a metallic layer in a vapor deposition process.

23. The method of claim 15, wherein the creating of the inductor comprises using chemical etching with a photolithographically patterned photoresist mask.

24. The method of claim 15, further comprising creating a capacitor comprising overlapping end sections of the inductor, causing the inductor to have an offset step in a thickness direction adjacent at least one of the end sections, and locating an intermediate dielectric portion of the polymeric material between the end sections to separate and insulate the end sections.

25. The method of claim 15, wherein the creating the three-dimensionally undulating shape further comprises thermally molding a metallic layer.

26. The method of claim 15, wherein the creating the three-dimensionally undulating shape further comprises compressing a metallic layer between mold halves of the mold, each including undulating cavities therein.

27. The method of claim 15, wherein the creating the three-dimensionally undulating shape further comprises:
creating a vacuum or inserting an inert gas in an oven within which a metallic layer is located to deter oxidation and brittleness of the metallic layer in the oven;
heating the metallic layer in an oven; and
creating three-dimensionally undulating peaks and valleys in the metallic layer in the oven.
